(19) United States
(12) Patent Application Publication
(10) Pub. No.: US 2021/0052165 A1
(43) Pub. Date: Feb. 25, 2021

(54) HEART RATE MEASUREMENT METHOD AND DEVICE, AND COMPUTER READABLE STORAGE MEDIUM

(71) Applicant: SHENZHEN YOLANDA TECHNOLOGY CO., LTD., Shenzhen (CN)

(72) Inventor: Jun CAO, Shenzhen (CN)

(73) Assignee: Shenzhen Youlanda Technology Co., Ltd., Shenzhen (CN)

(21) Appl. No.: 16/979,292

(22) PCT Filed: Sep. 27, 2018
(86) PCT No.: PCT/CN2018/107894

§ 371 Date: Sep. 9, 2020

(51) Int. Cl. A61B 5/024; A61B 5/00

(52) U.S. Cl. CPC A61B 5/024; A61B 5/7257

(57) ABSTRACT

The present disclosure provides a heart rate measurement method, including: using a human body parameter acquisition device to collect user data; obtaining a time domain heart rate value according to a time domain characteristic of the user data; performing frequency domain transformation on the user data to obtain user frequency domain data, and obtaining a frequency domain heart rate value according to the user frequency domain data; and obtaining a heart rate measurement value according to the time domain heart rate value and the frequency domain heart rate value. The present disclosure further provides a heart rate measurement device and a computer readable storage medium.

### TECHNICAL FIELD

The present disclosure relates to the technical field of physical sign monitoring, in particular to a heart rate measurement method and device, and computer readable storage medium.

### BACKGROUND

Heart rate, as an important indicator for measuring human health, has attracted more and more public attention. Nowadays, various electronic products that can measure heart rate are competing to enter consumers' sight. Most of these electronic products for measuring the heart rate calculate the heart rate based on the time domain information of the collected user data, but the heart rate value obtained from this calculation has a low accuracy.

The above content is only used to assist the understanding of the technical solution of the present disclosure, which does not mean that the above content is recognized as prior art.

### SUMMARY

The main objective of the present disclosure is to provide a heart rate measurement method and device, and a computer readable storage medium, aiming to solve the technical problem that the measurement accuracy of the heart rate is low.

In order to achieve the above objective, the present disclosure provides a heart rate measurement method, including:

using a human body parameter acquisition device to collect user data;

obtaining a time domain heart rate value according to a time domain characteristic of the user data;

performing frequency domain transformation on the user data to obtain user frequency domain data, and obtaining a frequency domain heart rate value according to the user frequency domain data; and

obtaining a heart rate measurement value according to the time domain heart rate value and the frequency domain heart rate value.

In an embodiment, the human body parameter acquisition device includes one or more of a weight measurement device, a height measurement device and a physical sign measurement device.

In an embodiment, the operation of obtaining a time domain heart rate value according to a time domain characteristic of the user data includes:

obtaining N time domain waveforms according to the user data, N being a positive integer; and

obtaining the time domain heart rate value according to the N time domain waveforms.

In an embodiment, the operation of obtaining the time domain heart rate value according to the N time domain waveforms includes:

determining whether a frequency of each of the N time domain waveforms meets a preset heart rate, and adding the time domain waveform that meets the preset heart rate to a first heart rate candidate set; and

obtaining the time domain heart rate value according to the first heart rate candidate set.

In an embodiment, the operation of obtaining the time domain heart rate value according to the N time domain waveforms includes:

determining whether a frequency of each of the N time domain waveforms meets a preset heart rate, and adding the time domain waveform that meets the preset heart rate to a first heart rate candidate set;

taking each time domain waveform in the first heart rate candidate set as a reference waveform, calculating a similarity between each of remaining time domain waveforms in the first heart rate candidate set and the reference waveform, and obtaining a number of time domain waveforms, the similarity between each of the obtained time domain waveforms and the reference waveform being greater than a preset similarity threshold;

taking a reference waveform corresponding to a maximum number of numbers and a time domain waveform as a second heart rate candidate set, the similarity between each of the time domain waveforms and the reference waveform corresponding to the maximum number of numbers being greater than the preset similarity threshold; and

obtaining the time domain heart rate value according to the second heart rate candidate set.

In an embodiment, a preset heart rate value is not less than 0.8 Hz and not more than 2.5 Hz.

In an embodiment, the operation of performing frequency domain transformation on the user data to obtain user frequency domain data, and obtaining a frequency domain heart rate value according to the user frequency domain data includes:

performing K fast Fourier transforms on the user data to obtain K spectrum analysis results, K being a positive integer;

obtaining K candidate frequency values according to the K spectrum analysis results; and

obtaining the frequency domain heart rate value according to the K candidate frequency values.

In an embodiment, the operation of obtaining a heart rate measurement value according to the time domain heart rate value and the frequency domain heart rate value includes:

determining a time domain weighting coefficient and a frequency domain weighting coefficient;

performing a time domain weighting operation on the time domain heart rate value according to the time domain weighting coefficient to obtain a time domain weighting heart rate value;

performing a frequency domain weighting operation on the frequency domain heart rate value according to the frequency domain weighting coefficient to obtain a frequency domain weighting heart rate value; and

obtaining the heart rate measurement value according to the time domain weighting heart rate value and the frequency domain weighting heart rate value.

In an embodiment, before the operation of obtaining a time domain heart rate value according to a time domain characteristic of the user data, the method further includes:

using a digital filter to smoothly filter the user data.

Besides, in order to achieve the above objective, the present disclosure further provides a heart rate measurement device, including a memory, a processor, a heart rate measurement program stored on the memory and executable on the processor, the heart rate measurement program, when executed by the processor, implements the following operations:

using a human body parameter acquisition device to collect user data;

obtaining a time domain heart rate value according to a time domain characteristic of the user data;

performing frequency domain transformation on the user data to obtain user frequency domain data, and obtaining a frequency domain heart rate value according to the user frequency domain data; and

obtaining a heart rate measurement value according to the time domain heart rate value and the frequency domain heart rate value.

In an embodiment, the human body parameter acquisition device includes one or more of a weight measurement device, a height measurement device and a physical sign measurement device.

In an embodiment, the heart rate measurement program, when executed by the processor, further implements the following operations:

obtaining N time domain waveforms according to the user data, N being a positive integer; and

obtaining the time domain heart rate value according to the N time domain waveforms.

In an embodiment, the heart rate measurement program, when executed by the processor, further implements the following operations:

determining whether a frequency of each of the N time domain waveforms meets a preset heart rate, and adding the time domain waveform that meets the preset heart rate to a first heart rate candidate set; and

obtaining the time domain heart rate value according to the first heart rate candidate set.

In an embodiment, the heart rate measurement program, when executed by the processor, further implements the following operations:

determining whether a frequency of each of the N time domain waveforms meets a preset heart rate, and adding the time domain waveform that meets the preset heart rate to a first heart rate candidate set;

taking each time domain waveform in the first heart rate candidate set as a reference waveform, calculating a similarity between each of remaining time domain waveforms in the first heart rate candidate set and the reference waveform, and obtaining a number of time domain waveforms, the similarity between each of the obtained time domain waveforms and the reference waveform being greater than a preset similarity threshold;

taking a reference waveform corresponding to a maximum number of numbers and a time domain waveform as a second heart rate candidate set, the similarity between each of the time domain waveforms and the reference waveform corresponding to the maximum number of numbers being greater than the preset similarity threshold; and

obtaining the time domain heart rate value according to the second heart rate candidate set.

In an embodiment, a preset heart rate value is not less than 0.8 Hz and not more than 2.5 Hz.

In an embodiment, the heart rate measurement program, when executed by the processor, further implements the following operations:

performing K fast Fourier transforms on the user data to obtain K spectrum analysis results, K being a positive integer;

obtaining K candidate frequency values according to the K spectrum analysis results; and

obtaining the frequency domain heart rate value according to the K candidate frequency values.

In an embodiment, the heart rate measurement program, when executed by the processor, further implements the following operations:

determining a time domain weighting coefficient and a frequency domain weighting coefficient;

performing a time domain weighting operation on the time domain heart rate value according to the time domain weighting coefficient to obtain a time domain weighting heart rate value;

performing a frequency domain weighting operation on the frequency domain heart rate value according to the frequency domain weighting coefficient to obtain a frequency domain weighting heart rate value; and

obtaining the heart rate measurement value according to the time domain weighting heart rate value and the frequency domain weighting heart rate value.

In an embodiment, before the operation of obtaining a time domain heart rate value according to a time domain characteristic of the user data, further implements the following operations:

using a digital filter to smoothly filter the user data.

Besides, in order to achieve the above objective, the present disclosure further provides a computer readable storage medium, wherein a heart rate measurement program is stored on the computer readable storage medium, the heart rate measurement program, when executed by a processor, implements the following operations:

using a human body parameter acquisition device to collect user data;

obtaining a time domain heart rate value according to a time domain characteristic of the user data;

performing frequency domain transformation on the user data to obtain user frequency domain data, and obtaining a frequency domain heart rate value according to the user frequency domain data; and

obtaining a heart rate measurement value according to the time domain heart rate value and the frequency domain heart rate value.

In an embodiment, the human body parameter acquisition device includes one or more of a weight measurement device, a height measurement device and a physical sign measurement device.

The present disclosure provides a heart rate measurement method, including: using a human body parameter acquisition device to collect user data; obtaining a time domain heart rate value according to a time domain characteristic of the user data; performing frequency domain transformation on the user data to obtain user frequency domain data, and obtaining a frequency domain heart rate value according to the user frequency domain data; and obtaining a heart rate measurement value according to the time domain heart rate value and the frequency domain heart rate value. Simple time domain analysis is difficult to filter out some periodic signal interference, but it can track the transient signal well. However, simple frequency domain analysis is more difficult to deal with the transient signal, shaking and other interference, and will introduce spectrum interference. But, frequency domain analysis has strong analysis ability in the environment with weak periodic signals, and can extract useful signals well. In the present disclosure, by using the complementarity of the time domain information and the frequency domain information of the user data, the calculation error of obtaining the heart rate value when using the time domain information or the frequency domain information alone is reduced, thereby improving the measurement accuracy of the heart rate value.

### BRIEF DESCRIPTION OF THE DRAWINGS

FIG. 1 is a schematic structural diagram of a terminal/device in a hardware operating environment according to an embodiment of the present disclosure.

FIG. 2 is a schematic flowchart of a heart rate measurement method according to a first embodiment of the present disclosure.

FIG. 3 is a schematic flowchart of the heart rate measurement method according to a second embodiment of the present disclosure.

FIG. 4 is a detailed flowchart of the operation of obtaining a time domain heart rate value according to a time domain characteristics of user data in the heart rate measurement method of the present disclosure.

FIG. 5 is another detailed flowchart of the operation of obtaining a time domain heart rate value according to a time domain characteristics of user data in the heart rate measurement method of the present disclosure.

FIG. 6 is a detailed flowchart of the operation of performing frequency domain transformation on the user data to obtain user frequency domain data, and obtaining a frequency domain heart rate value according to the user frequency domain data in the heart rate measurement method of the present disclosure.

FIG. 7 is a detailed flowchart of the operation of obtaining a heart rate measurement value according to the time domain heart rate value and the frequency domain heart rate value in the heart rate measurement method of the present disclosure.

The realization of the objective, functional characteristics, and advantages of the present disclosure are further described with reference to the accompanying drawings.

### DETAILED DESCRIPTION OF THE EMBODIMENTS

It should be understood that the specific embodiments described here are only used to explain the present disclosure, and are not intended to limit the present disclosure.

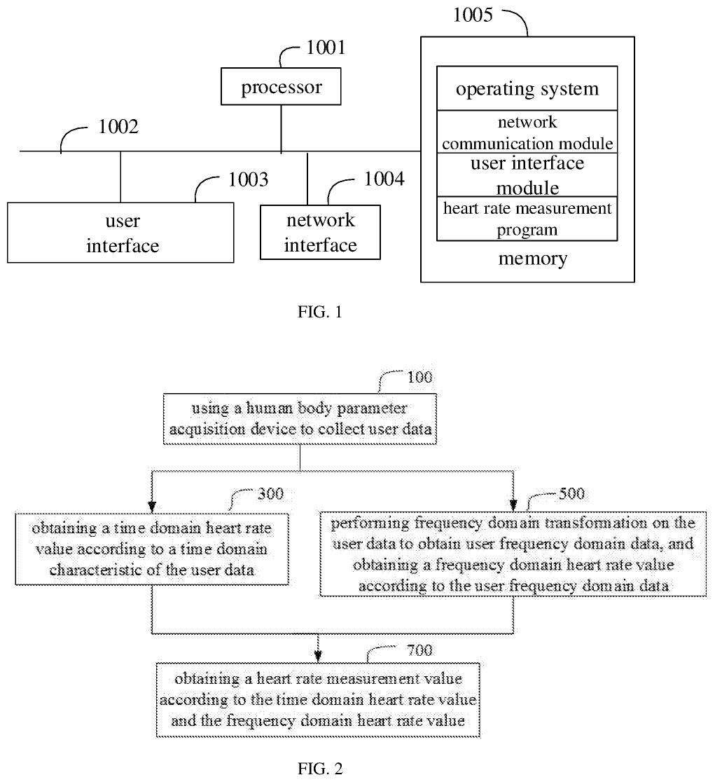

FIG. 1 is a schematic structural diagram of a terminal to which a heart rate measurement device in a hardware operating environment belongs according to an embodiment of the present disclosure.

The terminal in the embodiment of the present disclosure may be a PC, or a mobile terminal device with a display function such as a smart phone, a tablet computer, an e-book reader, a Moving Picture Experts Group Audio Layer III (MP3) player, a Moving Picture Experts Group Audio Layer IV (MP4) player, and a portable computer.

As shown in FIG. 1, the terminal may include: a processor 1001, such as a CPU, a network interface 1004, a user interface 1003, a memory 1005, and a communication bus 1002. The communication bus 1002 is configured to implement connection and communication between these components. The user interface 1003 may include a display and an input unit such as a keyboard. Optionally, the user interface 1003 may also include a standard wired interface and a wireless interface. The network interface 1004 may optionally include a standard wired interface and a wireless interface (such as a WI-FI interface). The memory 1005 may be a high-speed random access memory (RAM), or a stable memory (non-volatile memory), such as a magnetic disk memory. Optionally, the memory 1005 may also be a storage device independent of the foregoing processor 1001.

In an embodiment, the terminal may also include a camera, a Radio Frequency (RF) circuit, a sensor, an audio circuit, a WiFi module, and so on. The sensor may be, for example, a light sensor, a motion sensor, and other sensors. Specifically, the light sensor may include an ambient light sensor and a proximity sensor. The ambient light sensor can adjust the brightness of the display screen according to the brightness of the ambient light, and the proximity sensor can turn off the display screen and/or the backlight when the mobile terminal is moved to the ear. A gravity acceleration sensor, as a kind of motion sensor, may detect the magnitude of acceleration in all directions (usually three axes). The gravity acceleration sensor may detect the magnitude and direction of gravity when it is stationary, and may be configured to identify the gesture of the mobile terminal (such as horizontal and vertical screen switching, related games, magnetometer gesture calibration), vibration recognition related functions (such as pedometer, tapping), etc. Of course, the mobile terminal may also be equipped with other sensors such as a gyroscope, a barometer, a hygrometer, a thermometer, an infrared sensor, and the like, which will not be repeated here.

Those skilled in the art should understand that the terminal structure shown in FIG. 1 does not constitute a limitation on the terminal, and may include more or fewer components, a combination of some components, or differently arranged components than shown in the figure.

As shown in FIG. 1, a memory 1005 as a computer storage medium may include an operating system, a network communication module, a user interface module, and a heart rate measurement program.

In the terminal shown in FIG. 1, the network interface 1004 is mainly configured to connect to a background server and perform data communication with the background server. The user interface 1003 is mainly configured to connect to a client (user) and perform data communication with the client. The processor 1001 may be configured to call the heart rate measurement program stored on the memory 1005.

In this embodiment, the heart rate measurement device includes the memory 1005, the processor 1001, and a heart rate measurement program stored on the memory 1005 and executable on the processor 1001. The processor 1001 may call the heart rate measurement program stored on the memory 1005, and perform the following operations:

using a human body parameter acquisition device to collect user data;

obtaining a time domain heart rate value according to a time domain characteristic of the user data;

performing frequency domain transformation on the user data to obtain user frequency domain data, and obtaining a frequency domain heart rate value according to the user frequency domain data; and

obtaining a heart rate measurement value according to the time domain heart rate value and the frequency domain heart rate value.

In an embodiment, the human body parameter acquisition device includes one or more of a weight measurement device, a height measurement device and a physical sign measurement device.

In an embodiment, the processor 1001 may call the heart rate measurement program stored on the memory 1005, and perform the following operations:

using a digital filter to smoothly filter the user data.

In an embodiment, the processor 1001 may call the heart rate measurement program stored on the memory 1005, and perform the following operations:

obtaining N time domain waveforms according to the user data, N being a positive integer; and

obtaining the time domain heart rate value according to the N time domain waveforms.

In an embodiment, the processor 1001 may call the heart rate measurement program stored on the memory 1005, and perform the following operations:

determining whether a frequency of each of the N time domain waveforms meets a preset heart rate, and adding the time domain waveform that meets the preset heart rate to a first heart rate candidate set; and

obtaining the time domain heart rate value according to the first heart rate candidate set.

In an embodiment, the processor 1001 may call the heart rate measurement program stored on the memory 1005, and perform the following operations:

determining whether a frequency of each of the N time domain waveforms meets a preset heart rate, and adding the time domain waveform that meets the preset heart rate to a first heart rate candidate set;

taking each time domain waveform in the first heart rate candidate set as a reference waveform, calculating a similarity between each of remaining time domain waveforms in the first heart rate candidate set and the reference waveform, and obtaining a number of time domain waveforms, the similarity between the time domain waveform and the reference waveform being greater than a preset similarity threshold;

taking the reference waveform corresponding to a maximum number of numbers and the time domain waveform as a second heart rate candidate set, the similarity between the time domain waveform and the reference waveform corresponding to the maximum number of numbers being greater than the preset similarity threshold; and

obtaining the time domain heart rate value according to the second heart rate candidate set.

In an embodiment, a preset heart rate value is not less than 0.8 Hz and not more than 2.5 Hz.

In an embodiment, the processor 1001 may call the heart rate measurement program stored on the memory 1005, and perform the following operations:

performing K fast Fourier transforms on the user data to obtain K spectrum analysis results, K being a positive integer;

obtaining K candidate frequency values according to the K spectrum analysis results; and

obtaining the frequency domain heart rate value according to the K candidate frequency values.

In an embodiment, the processor 1001 may call the heart rate measurement program stored on the memory 1005, and perform the following operations:

determining a time domain weighting coefficient and a frequency domain weighting coefficient;

performing a time domain weighting operation on the time domain heart rate value according to the time domain weighting coefficient to obtain a time domain weighting heart rate value;

performing a frequency domain weighting operation on the frequency domain heart rate value according to the frequency domain weighting coefficient to obtain a frequency domain weighting heart rate value; and

obtaining the heart rate measurement value according to the time domain weighting heart rate value and the frequency domain weighting heart rate value.

In an embodiment, the processor 1001 may call the heart rate measurement program stored on the memory 1005, and perform the following operations:

defining user data buffer space configured to store collected user data, time domain data buffer space configured to store data generated during the process of obtaining time domain heart rate values, and frequency domain data buffer space configured to store data generated during the process of obtaining heart rate measurement values.

A first embodiment of the present disclosure provides a heart rate measurement method. As shown in FIG. 2, FIG. 2 is a schematic flowchart of the heart rate measurement method according to a first embodiment of the present disclosure. The method includes the following operations.

S100, using a human body parameter acquisition device to collect user data.

Specially, a weight measurement device, a height measurement device, or a physical sign measurement device can be used to collect user data such as bioelectrical impedance signals or electrical potential difference signals through contact or non-contact sensing of the user's skin. These user data have a certain corresponding relationship with the human heart rate. In some embodiments, the physical sign measurement device includes a smart bracelet, a smart watch, etc., which will not be repeated in the present disclosure.

Take the use of the weight measurement device (such as a body scale) to collect user data as an example for description. When the user is standing on the weight measurement device, the weight measurement device can collect user data, such as the bioelectrical impedance signal or electric potential difference signal of the user's sole, through the part of the scale surface in contact with the skin of the user's sole. The measurement of bioelectrical impedance signal is based on the conductivity of body tissues when high-frequency, low-intensity alternating current flows through the human body for a short time. One or more bioelectrical impedance sensors are provided on the weight measurement device. When the user stands on the weight measurement device, the bioelectrical impedance sensor collects the bioelectrical impedance signal of the user's sole.

S300, obtaining a time domain heart rate value according to a time domain characteristic of the user data.

Specially, the waveform signal in the time domain is obtained according to the collected user data. Since any two adjacent peaks or any two adjacent troughs in the waveform signal correspond to a heartbeat cycle, the time domain heart rate value can be calculated according to the heartbeat cycle.

For example, firstly N time domain waveforms (N is a positive integer) is obtained according to user data, and then the time domain heart rate value is obtained according to the N time domain waveforms. It is easy to understand that the larger the value of N, the more data used to calculate the time domain heart rate value, and the higher the accuracy of the calculated time domain heart rate value. But at the same time, it may be at the cost of reducing the processing efficiency. Therefore, the specific value of N should be determined according to the actual situation.

S500, performing frequency domain transformation on the user data to obtain user frequency domain data, and obtaining a frequency domain heart rate value according to the user frequency domain data.

Specially, the user frequency domain data is obtained through performing fast Fourier transform on the user data.

S700, obtaining a heart rate measurement value according to the time domain heart rate value and the frequency domain heart rate value.

In operation S300 and operation S500, the time domain heart rate value and the frequency domain heart rate value are obtained respectively. In operation S700, according to the reliability of the time domain heart rate value and the frequency domain heart rate value, different weighting coefficients can be used to calculate the heart rate measurement value with comprehensive evaluation value.

Since there is a distance between the surface of the human body and the heart of the human body, the signal that can reflect the heart rate value measured at the surface of the human body (such as the skin on the sole of the foot) is relatively weak, and the user may experience jitter or shaking during the measurement, thus the user data collected by the existing heart rate measurement method often contains interference signals, resulting in low accuracy of heart rate measurement.

The present disclosure provides a heart rate measurement method, including: using a human body parameter acquisition device to collect user data; obtaining a time domain heart rate value according to a time domain characteristic of the user data; performing frequency domain transformation on the user data to obtain user frequency domain data, and obtaining a frequency domain heart rate value according to the user frequency domain data; and obtaining a heart rate measurement value according to the time domain heart rate value and the frequency domain heart rate value. Simple time domain analysis is difficult to filter out some periodic signal interference, but it can track the transient signal well. In the present disclosure, by using the complementarity of the time domain information and the frequency domain information of the user data, the calculation error of obtaining the heart rate value when using the time domain information or the frequency domain information alone is reduced, thereby improving the measurement accuracy of the heart rate value.

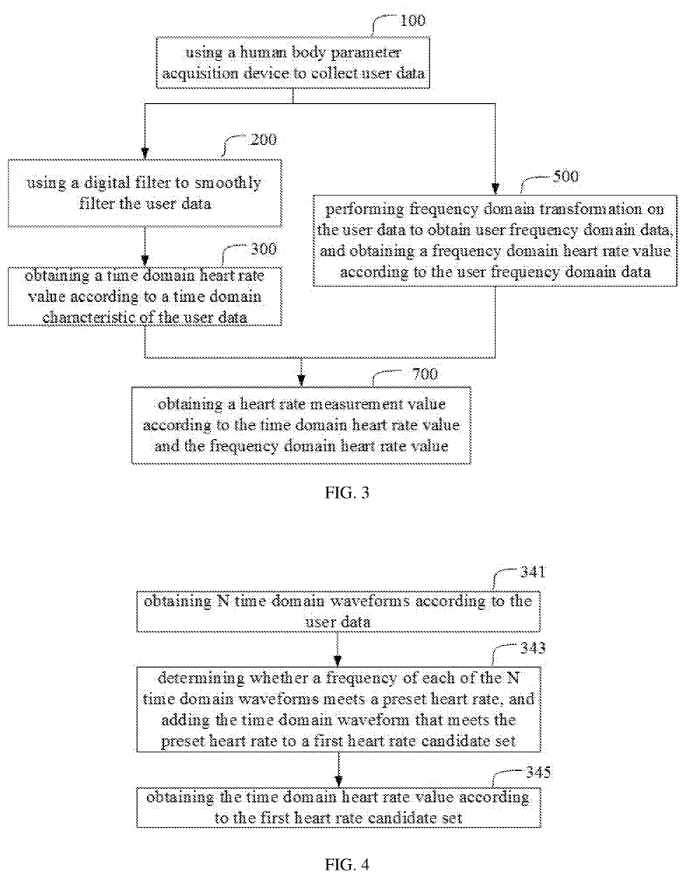

Based on the first embodiment, a second embodiment of the heart rate measurement method of the present disclosure is proposed. As shown in FIG. 3, before operation S300, the method further includes:

S200, using a digital filter to smoothly filter user data.

Specially, a digital filter is used to smoothly filter the collected user data to track and filter out the transient signal in the user data. After the above operations, the time domain heart rate value is obtained according to the time domain characteristics of the user data after the smoothing and filtering process. In an embodiment, a FIR digital filter is used to smoothly filter the user data.

Based on the first embodiment, a third embodiment of the heart rate measurement method of the present disclosure is proposed. As shown in FIG. 4, before operation S300, the method further includes:

S341, obtaining N time domain waveforms according to the user data.

Specially, the body scale samples the user's sole signal through a preset sampling frequency to obtain an amount of user data, obtains the time domain waveforms of the user data in the time domain, and extracts N time domain waveforms from the above-mentioned time domain waveforms.

S343, determining whether a frequency of each of the N time domain waveforms meets a preset heart rate, and adding the time domain waveform that meets the preset heart rate to a first heart rate candidate set

In an embodiment, a preset heart rate value is not less than 0.8 Hz and not more than 2.5 Hz. Specially, operation S333 includes:

S3431, obtaining an empty first heart rate candidate set;

S3433, processing each of the N time domain waveforms;

determining whether the frequency of the i-th time domain waveform is within the preset heart rate range, if so, adding the i-th time domain waveform to the first heart rate candidate set;

if not, then skipping to add the i-th time domain waveform to the first heart rate candidate set;

i is not less than 1 and not more than N.

The first heart rate candidate set is obtained by determining the frequency of each time domain waveform in the N time domain waveforms, and the frequency of each time domain waveform in the first heart rate candidate set meets the preset heart rate.

S345, obtaining the time domain heart rate value according to the first heart rate candidate set.

Assuming that the first heart rate candidate set includes N1 time domain waveforms, the time domain heart rate value is calculated according to the time domain characteristics of the above N1 time domain waveforms, N1 is a positive integer and N1 is not greater than N.

In an embodiment, the frequency of each time domain waveform in the first heart rate candidate set is calculated separately to obtain N1 time domain frequency values, and then the time domain heart rate value is calculated according to the average value of the above-mentioned N1 time domain frequency values.

In this embodiment, firstly, N time domain waveforms is obtained according to user data; secondly, by determining whether the frequency of the N time domain waveforms meets the preset heart rate, all time domain waveforms that meet the preset heart rate are selected to form the first heart rate candidate set; finally, the time domain heart rate value is calculated according to the first heart rate candidate set. By filtering the time domain waveform on the time domain characteristics, it can filter out the shaking or some periodic weak signal interference during the measurement, and better extract the effective heart rate signal, thereby improving the measurement accuracy of the time domain heart rate value.

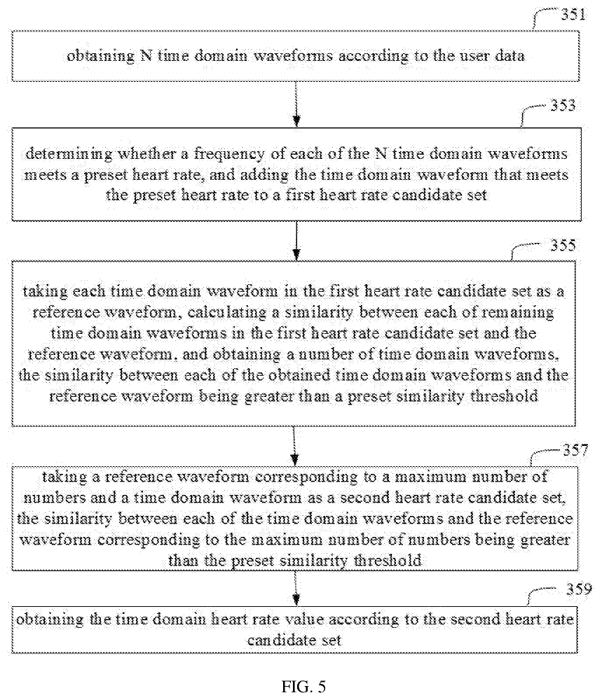

Based on the first embodiment, a fourth embodiment of the heart rate measurement method of the present disclosure is proposed. As shown in FIG. 5, operation S300 includes:

S351, obtaining N time domain waveforms according to the user data.

Specially, the body scale samples the user's sole signal through a preset sampling frequency to obtain an amount of user data, obtains the time domain waveforms of the user data in the time domain, and extracts N time domain waveforms from the above-mentioned time domain waveforms.

S353, determining whether a frequency of each of the N time domain waveforms meets a preset heart rate, and adding the time domain waveform that meets the preset heart rate to a first heart rate candidate set

In an embodiment, a preset heart rate value is not less than 0.8 Hz and not more than 2.5 Hz. Specially, operation S343 includes:

S3531, obtaining an empty first heart rate candidate set;

S3533, processing each of the N time domain waveforms;

determining whether the frequency of the i-th time domain waveform is within the preset heart rate range, if so, adding the i-th time domain waveform to the first heart rate candidate set;

if not, then skipping to add the i-th time domain waveform to the first heart rate candidate set;

i is not less than 1 and not more than N.

The first heart rate candidate set is obtained by determining the frequency of each time domain waveform in the N time domain waveforms, and the frequency of each time domain waveform in the first heart rate candidate set meets the preset heart rate.

S355, taking each time domain waveform in the first heart rate candidate set as a reference waveform, calculating a similarity between each of remaining time domain waveforms in the first heart rate candidate set and the reference waveform, and obtaining a number of time domain waveforms, the similarity between the time domain waveform and the reference waveform being greater than a preset similarity threshold.

When the reference waveform is selected and other time domain waveforms similar to the reference waveform are obtained by traversing the first heart rate candidate set, the similarity can be determined through the period and/or amplitude of the reference waveform and the time domain waveform to be compared. If the similarity is greater than the preset similarity threshold, the reference waveform and the time domain waveform to be compared are similar; otherwise, the reference waveform and the time domain waveform to be compared are not similar.

Specially, assuming that the first heart rate candidate set includes N1 time domain waveforms, N1 is a positive integer and N1 is not greater than N, operation S355 includes:

S3551, processing each time domain waveform in the first heart rate candidate set.

let the j-th time domain waveform be the reference waveform, and let the initial value of the number of similar waveforms Num(j) of the reference waveform be 0, and the initial value of the set of similar waveforms Wave(j) of the reference waveform be empty;

traverse the first heart rate candidate set to obtain the similarity between the k-th time domain waveform and the reference waveform, if the similarity is greater than the preset similarity threshold, the k-th time domain waveform is added to the set of similar waveforms Wave(j) of the reference waveform, and the number of similar waveforms of the reference waveform is increased by one, that is, Num(j)=Num(j)+1, j and k are not less than 1 and not greater than N1, and j is not equal to k.

S3553, obtaining the set of similar waveforms {Wave(1), . . . , Wave(j), . . . , Wave(N1)} and the number of similar waveforms {Num (1), . . . , Num (j), . . . , Num(N1)} of each time domain waveform in the first heart rate candidate set.

S357, taking the reference waveform corresponding to a maximum number of numbers and the time domain waveform as a second heart rate candidate set, the similarity between the time domain waveform and the reference waveform corresponding to the maximum number of numbers being greater than the preset similarity threshold.

Specially, according to the number of similar waveforms {Num (1), . . . , Num (j), Num(N1)} in each time domain waveform in the first heart rate candidate set, select the time domain waveform corresponding to the maximum value. For example, if the p-th time domain waveform has the largest number of similar waveforms, the set of similar waveforms Wave(p) corresponding to the p-th time domain waveform is obtained, the p-th time domain waveform and each time domain waveform in the corresponding similar waveform set Wave(p) together constitute the second heart rate candidate set; p is not less than 1 and not greater than N1.

S359, obtaining the time domain heart rate value according to the second heart rate candidate set.

Assuming that the second heart rate candidate set includes N2 time domain waveforms, the time domain heart rate value is calculated according to the time domain characteristics of the above-mentioned N2 time domain waveforms; N2 is a positive integer and N2 is not greater than N1.

In an embodiment, the frequency of each time domain waveform in the second heart rate candidate set is calculated separately to obtain N2 time domain frequency values, and then the time domain heart rate value is calculated according to the average value of the aforementioned N2 time domain frequency values.

In this embodiment, firstly, N time domain waveforms are obtained based on user data, and secondly, by determining whether the frequency of the N time domain waveforms meets the preset heart rate, all time domain waveforms that meet the preset heart rate are selected to form the first heart rate candidate set. Then, according to the similarity between each time domain waveform in the first heart rate candidate set, the group of time domain waveforms with the most similar waveforms is further selected to form the second heart rate candidate set. Finally, the time domain heart rate value is calculated according to the second heart rate candidate set. Through the secondary selection of the time domain waveform on the time domain characteristics, the shaking or the interference of some periodic weak small signals during measurement is further filtered out, better extracting the effective heart rate signal, thereby improving the measurement accuracy of the time domain heart rate value.

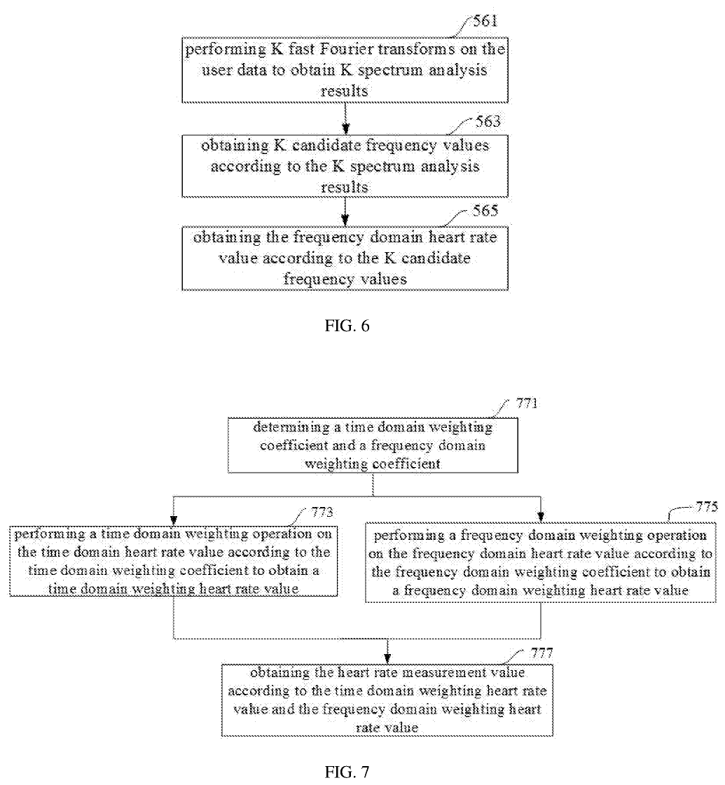

Based on the first embodiment, a fifth embodiment of the heart rate measurement method of the present disclosure is proposed. As shown in FIG. 6, operation S500 includes:

S561, performing K fast Fourier transforms on the user data to obtain K spectrum analysis results, K being a positive integer.

Assuming that the length of the user data is Len, the subscript of the user data is 0˜(Len−1). Specifically, when performing the k-th fast Fourier transform on the user data, perform the fast Fourier transform on the data whose subscripts are n˜Len−1; n is not less than 1 and not greater than (Len−1), k is not less than 1 and not greater than K.

S563, obtaining K candidate frequency values according to the K spectrum analysis results.

Specifically, each of the K spectrum analysis results is processed.

In the k-th spectrum analysis result, the frequency corresponding to the frequency point with the largest energy and multiplier is taken as the k-th candidate frequency value f(k).

S565, obtaining the frequency domain heart rate value according to the K candidate frequency values.

After the processing of operation S553, a set of K candidate frequency values {f(1), . . . , f(k), . . . , f(K)} is obtained, and the frequency with the most occurrences in the set of candidate frequency values is used as the frequency domain heart rate value. The number of occurrences of the frequency selected as the frequency domain heart rate value in the candidate frequency value set is denoted as K1.

In this embodiment, firstly, K fast Fourier transforms are performed on user data to obtain the corresponding K spectrum analysis results. Secondly, the corresponding K candidate frequency values are obtained according to the K spectrum analysis results. Finally, the frequency domain heart rate value is obtained according to the K candidate frequency values. By processing the spectrum analysis results of the user data after multiple fast Fourier transforms, the noise caused by the entire spectrum analysis due to the external interference of a segment of data can be effectively filtered out.

Based on the first embodiment, a sixth embodiment of the heart rate measurement method of the present disclosure is proposed. As shown in FIG. 7, operation S700 includes:

S771, determining a time domain weighting coefficient and a frequency domain weighting coefficient.

Specifically, the reliability of the time domain heart rate value and the frequency domain heart rate value are respectively determined, so that the heart rate value with greater reliability corresponds to a larger weighting coefficient. Conversely, the heart rate value with a lower reliability corresponds to a smaller weighting coefficient.

For example, in operation S30, the number of acquired time domain waveforms in the first heart rate candidate set is N1. In operation S50, it is obtained that the number of occurrences of the frequency value selected as the frequency domain heart rate value in the candidate frequency value set is K1. Then, the time domain weighting coefficient and the frequency domain weighting coefficient can be determined according to the relative size of N1/N and K1/K; or

In operation S30, the number of acquired time domain waveforms in the second heart rate candidate set is N2. In operation S50, it is obtained that the number of occurrences of the frequency value selected as the frequency domain heart rate value in the candidate frequency value set is K1. Then, the time domain weighting coefficient and the frequency domain weighting coefficient can be determined according to the relative size of N2/N and K1/K.

S773, performing a time domain weighting operation on the time domain heart rate value according to the time domain weighting coefficient to obtain a time domain weighting heart rate value.

Specifically, the product of the time domain weighting coefficient and the time domain heart rate value is used as the time domain weighting heart rate value.

S775, performing a frequency domain weighting operation on the frequency domain heart rate value according to the frequency domain weighting coefficient to obtain a frequency domain weighting heart rate value.

Specifically, the product of the frequency domain weighting coefficient and the frequency domain heart rate value is used as the frequency domain weighting heart rate value.

S777, obtaining the heart rate measurement value according to the time domain weighting heart rate value and the frequency domain weighting heart rate value.

In an embodiment, the heart rate measurement value is calculated by summing the time domain weighting heart rate value and the frequency domain weighting heart rate value.

In this embodiment, the heart rate measurement value is obtained by weighting the time domain heart rate value and the frequency domain heart rate value, so that the result of the heart rate measurement value is more effective and reasonable.

Based on the first embodiment, a seventh embodiment of the heart rate measurement method of the present disclosure is proposed. Before operation S10, the method further includes:

defining user data buffer space configured to store collected user data, time domain data buffer space configured to store data generated during the process of obtaining time domain heart rate values, and frequency domain data buffer space configured to store data generated during the process of obtaining heart rate measurement values.

Specifically, a memory of a specified size (LEN) may be defined as the user data buffer space, a memory of a specified size (LEN) may be defined as the time domain data buffer space, and a memory of a specified size (4*LEN) may be defined as the frequency domain data buffer space.

Besides, the present disclosure further provides a computer readable storage medium. A heart rate measurement program is stored on the computer readable storage medium, the heart rate measurement program, when executed by a processor, implements the following operations:

using a human body parameter acquisition device to collect user data;

obtaining a time domain heart rate value according to a time domain characteristic of the user data;

performing frequency domain transformation on the user data to obtain user frequency domain data, and obtaining a frequency domain heart rate value according to the user frequency domain data; and

obtaining a heart rate measurement value according to the time domain heart rate value and the frequency domain heart rate value.

In an embodiment, the human body parameter acquisition device includes one or more of a weight measurement device, a height measurement device or a physical sign measurement device.

In an embodiment, the heart rate measurement program, when executed by a processor, implements the following operations:

using a digital filter to smoothly filter the user data.

In an embodiment, the heart rate measurement program, when executed by a processor, implements the following operations:

obtaining N time domain waveforms according to the user data, N being a positive integer; and

obtaining the time domain heart rate value according to the N time domain waveforms.

In an embodiment, the heart rate measurement program, when executed by a processor, implements the following operations:

determining whether a frequency of each of the N time domain waveforms meets a preset heart rate, and adding the time domain waveform that meets the preset heart rate to a first heart rate candidate set; and

obtaining the time domain heart rate value according to the first heart rate candidate set.

In an embodiment, the heart rate measurement program, when executed by a processor, implements the following operations:

determining whether a frequency of each of the N time domain waveforms meets a preset heart rate, and adding the time domain waveform that meets the preset heart rate to a first heart rate candidate set;

taking each time domain waveform in the first heart rate candidate set as a reference waveform, calculating a similarity between each of remaining time domain waveforms in the first heart rate candidate set and the reference waveform, and obtaining a number of time domain waveforms, the similarity between the time domain waveform and the reference waveform being greater than a preset similarity threshold;

taking the reference waveform corresponding to a maximum number of numbers and the time domain waveform as a second heart rate candidate set, the similarity between the time domain waveform and the reference waveform corresponding to the maximum number of numbers being greater than the preset similarity threshold; and

obtaining the time domain heart rate value according to the second heart rate candidate set.

In an embodiment, a preset heart rate value is not less than 0.8 Hz and not more than 2.5 Hz.

In an embodiment, the heart rate measurement program, when executed by a processor, implements the following operations:

performing K fast Fourier transforms on the user data to obtain K spectrum analysis results, K being a positive integer;

obtaining K candidate frequency values according to the K spectrum analysis results; and

obtaining the frequency domain heart rate value according to the K candidate frequency values.

In an embodiment, the heart rate measurement program, when executed by a processor, implements the following operations:

determining a time domain weighting coefficient and a frequency domain weighting coefficient;

performing a time domain weighting operation on the time domain heart rate value according to the time domain weighting coefficient to obtain a time domain weighting heart rate value;

performing a frequency domain weighting operation on the frequency domain heart rate value according to the frequency domain weighting coefficient to obtain a frequency domain weighting heart rate value; and

obtaining the heart rate measurement value according to the time domain weighting heart rate value and the frequency domain weighting heart rate value.

In an embodiment, the heart rate measurement program, when executed by a processor, implements the following operations:

defining user data buffer space configured to store collected user data, time domain data buffer space configured to store data generated during the process of obtaining time domain heart rate values, and frequency domain data buffer space configured to store data generated during the process of obtaining heart rate measurement values.

It should be noted that in this document, the terms “comprise”, “include” or any other variants thereof are intended to cover a non-exclusive inclusion. Thus, a process, method, article, or system that includes a series of elements not only includes those elements, but also includes other elements that are not explicitly listed, or also includes elements inherent to the process, method, article, or system. If there are no more restrictions, the element defined by the sentence “including a . . . ” does not exclude the existence of other

What is claimed is:

1. A heart rate measurement method, comprising the following operations:
using a human body parameter acquisition device to collect user data;
obtaining a time domain heart rate value according to a time domain characteristic of the user data;
performing frequency domain transformation on the user data to obtain user frequency domain data, and obtaining a frequency domain heart rate value according to the user frequency domain data; and
obtaining a heart rate measurement value according to the time domain heart rate value and the frequency domain heart rate value.

2. The heart rate measurement method of claim 1, wherein the human body parameter acquisition device comprises one or more of a weight measurement device, a height measurement device and a physical sign measurement device.

3. The heart rate measurement method of claim 1, wherein the operation of obtaining a time domain heart rate value according to a time domain characteristic of the user data comprises:
obtaining N time domain waveforms according to the user data, N being a positive integer; and
obtaining the time domain heart rate value according to the N time domain waveforms.

4. The heart rate measurement method of claim 3, wherein the operation of obtaining the time domain heart rate value according to the N time domain waveforms comprises:
determining whether a frequency of each of the N time domain waveforms meets a preset heart rate, and adding the time domain waveform that meets the preset heart rate to a first heart rate candidate set; and
obtaining the time domain heart rate value according to the first heart rate candidate set.

5. The heart rate measurement method of claim 3, wherein the operation of obtaining the time domain heart rate value according to the N time domain waveforms comprises:
determining whether a frequency of each of the N time domain waveforms meets a preset heart rate, and adding the time domain waveform that meets the preset heart rate to a first heart rate candidate set;
taking each time domain waveform in the first heart rate candidate set as a reference waveform, calculating a similarity between each of remaining time domain waveforms in the first heart rate candidate set and the reference waveform, and obtaining a number of time domain waveforms, the similarity between each of the obtained time domain waveforms and the reference waveform being greater than a preset similarity threshold;
taking a reference waveform corresponding to a maximum number of numbers and a time domain waveform as a second heart rate candidate set, the similarity between each of the time domain waveforms and the reference waveform corresponding to the maximum number of numbers being greater than the preset similarity threshold; and
obtaining the time domain heart rate value according to the second heart rate candidate set.

6. The heart rate measurement method of claim 4, wherein a preset heart rate value is not less than 0.8 Hz and not more than 2.5 Hz.

7. The heart rate measurement method of claim 1, wherein the operation of performing frequency domain transformation on the user data to obtain user frequency domain data, and obtaining a frequency domain heart rate value according to the user frequency domain data comprises:
performing K fast Fourier transforms on the user data to obtain K spectrum analysis results, K being a positive integer;
obtaining K candidate frequency values according to the K spectrum analysis results; and
obtaining the frequency domain heart rate value according to the K candidate frequency values.

8. The heart rate measurement method of claim 1, wherein the operation of obtaining a heart rate measurement value according to the time domain heart rate value and the frequency domain heart rate value comprises:
determining a time domain weighting coefficient and a frequency domain weighting coefficient;
performing a time domain weighting operation on the time domain heart rate value according to the time domain weighting coefficient to obtain a time domain weighting heart rate value;
performing a frequency domain weighting operation on the frequency domain heart rate value according to the frequency domain weighting coefficient to obtain a frequency domain weighting heart rate value; and
obtaining the heart rate measurement value according to the time domain weighting heart rate value and the frequency domain weighting heart rate value.

9. The heart rate measurement method of claim 1, wherein before the operation of obtaining a time domain heart rate value according to a time domain characteristic of the user data, the method further comprises:
using a digital filter to smoothly filter the user data.

10. A heart rate measurement device, comprising a memory, a processor, a heart rate measurement program stored on the memory and executable on the processor, the heart rate measurement program, when executed by the processor, implements the following operations:
using a human body parameter acquisition device to collect user data;
obtaining a time domain heart rate value according to a time domain characteristic of the user data;
performing frequency domain transformation on the user data to obtain user frequency domain data, and obtaining a frequency domain heart rate value according to the user frequency domain data; and
obtaining a heart rate measurement value according to the time domain heart rate value and the frequency domain heart rate value.

11. The heart rate measurement device of claim 10, wherein the human body parameter acquisition device comprises one or more of a weight measurement device, a height measurement device and a physical sign measurement device.

12. The heart rate measurement device of claim 10, wherein the heart rate measurement program, when executed by the processor, further implements the following operations:
obtaining N time domain waveforms according to the user data, N being a positive integer; and
obtaining the time domain heart rate value according to the N time domain waveforms.

13. The heart rate measurement device of claim 12, wherein the heart rate measurement program, when executed by the processor, further implements the following operations:
determining whether a frequency of each of the N time domain waveforms meets a preset heart rate, and adding the time domain waveform that meets the preset heart rate to a first heart rate candidate set; and
obtaining the time domain heart rate value according to the first heart rate candidate set.

14. The heart rate measurement device of claim 12, wherein the heart rate measurement program, when executed by the processor, further implements the following operations:
determining whether a frequency of each of the N time domain waveforms meets a preset heart rate, and adding the time domain waveform that meets the preset heart rate to a first heart rate candidate set;
taking each time domain waveform in the first heart rate candidate set as a reference waveform, calculating a similarity between each of remaining time domain waveforms in the first heart rate candidate set and the reference waveform, and obtaining a number of time domain waveforms, the similarity between each of the obtained time domain waveforms and the reference waveform being greater than a preset similarity threshold;
taking a reference waveform corresponding to a maximum number of numbers and a time domain waveform as a second heart rate candidate set, the similarity between each of the time domain waveforms and the reference waveform corresponding to the maximum number of numbers being greater than the preset similarity threshold; and
obtaining the time domain heart rate value according to the second heart rate candidate set.

15. The heart rate measurement device of claim 13, wherein a preset heart rate value is not less than 0.8 Hz and not more than 2.5 Hz.

16. The heart rate measurement device of claim 10, wherein the heart rate measurement program, when executed by the processor, further implements the following operations:
performing K fast Fourier transforms on the user data to obtain K spectrum analysis results, K being a positive integer;
obtaining K candidate frequency values according to the K spectrum analysis results; and
obtaining the frequency domain heart rate value according to the K candidate frequency values.

17. The heart rate measurement device of claim 10, wherein the heart rate measurement program, when executed by the processor, further implements the following operations:
determining a time domain weighting coefficient and a frequency domain weighting coefficient;
performing a time domain weighting operation on the time domain heart rate value according to the time domain weighting coefficient to obtain a time domain weighting heart rate value;
performing a frequency domain weighting operation on the frequency domain heart rate value according to the frequency domain weighting coefficient to obtain a frequency domain weighting heart rate value; and
obtaining the heart rate measurement value according to the time domain weighting heart rate value and the frequency domain weighting heart rate value.

18. The heart rate measurement device of claim 10, wherein before the operation of obtaining a time domain heart rate value according to a time domain characteristic of the user data, further implements the following operations:
using a digital filter to smoothly filter the user data.

19. A computer readable storage medium, wherein a heart rate measurement program is stored on the computer readable storage medium, the heart rate measurement program, when executed by a processor, implements the following operations:
using a human body parameter acquisition device to collect user data;
obtaining a time domain heart rate value according to a time domain characteristic of the user data;
performing frequency domain transformation on the user data to obtain user frequency domain data, and obtaining a frequency domain heart rate value according to the user frequency domain data; and
obtaining a heart rate measurement value according to the time domain heart rate value and the frequency domain heart rate value.

20. The computer readable storage medium of claim 19, wherein the human body parameter acquisition device comprises one or more of a weight measurement device, a height measurement device and a physical sign measurement device.
